(19) United States
(12) Patent Application Publication
(10) Pub. No.: US 2004/0147994 A1
(43) Pub. Date: Jul. 29, 2004

(54) Tachy lead system optimized for septal placement

(75) Inventors: Yongxing Zhang, Little Canada, MN (US); Jeffrey P. Bodner, Roseville, MN (US)

Correspondence Address: SCHWEGMAN, LUNDBERG, WOESSNER & KLUTH, P.A., P.O. BOX 2938, MINNEAPOLIS, MN, 55402, US

(73) Assignee: Cardiac Pacemakers, Inc.

(21) Appl. No.: 10/352,546

(22) Filed: Jan. 28, 2003

(51) Int. Cl. A61N 1/05

(52) U.S. Cl. 607/127

(57) ABSTRACT

A lead assembly that includes a body with a proximal end and a distal end and at least one conductor disposed within the body. A distal electrode is at the distal end of the body and is electrically coupled with at least one of the conductors. A first electrode is also on the body and electrically coupled with at least one of the conductors. The lead assembly further includes a neck that is less stiff than the body. The neck is between the distal end of the body and the first electrode assembly. A fixation mechanism at the distal end of the body is capable of securing the lead assembly to a septum in a heart. A method is provided which includes advancing a lead assembly using a catheter until the lead assembly is near tissue, such as a septum in a heart. The lead assembly includes a body and a neck that is less stiff than the body. The method further includes affixing a distal end of the body to the septum, and removing the catheter to allow at least a portion of the neck to drop against the septum.

### TECHNICAL FIELD

The present invention relates generally to a lead for stimulating or monitoring tissue. More particularly, it pertains to a lead that provides ventricular pacing and/or sensing.

### BACKGROUND

Tachyarrhythmias are abnormal heart rhythms characterized by a rapid heart rate. Examples of tachyarrhythmias include supraventricular tachycardias (SVT's) such as sinus tachycardia, atrial tachycardia, and atrial fibrillation. The most dangerous tachyarrhythmias, however, are ventricular tachycardia (VT) and ventricular fibrillation (VF). Ventricular rhythms typically result in rapid and irregular contraction of the ventricles out of electromechanical synchrony with the atria.

Cardioversion and defibrillation can be used to terminate most tachyarrhythmias, including SVT's, VT, and VF. A class of cardiac rhythm management devices known as an implantable cardioverter/defibrillator (ICD) provides therapy by delivering a shock pulse to the heart, such as when the device detects fibrillation.

Another type of electrical therapy for tachycardia is antitachycardia pacing (ATP). In ATP, the heart is paced using one or more pacing pulses in an effort to correct the tachycardia. Modem ICD's typically have ATP capability so that ATP therapy is delivered to the heart when a tachycardia is detected, while a shock pulse is delivered when fibrillation occurs.

Although cardioversion/defibrillation will terminate tachycardia, it consumes a large amount of stored power from the battery and results in patient discomfort owing to the high voltage of the shock pulses. Therefore, it is desirable for the ICD to use ATP to terminate a tachyarrhythmia whenever possible.

ICD's typically include one or more leads that are implanted in or about the heart to reverse certain life threatening arrhythmias, or to stimulate contraction of the heart. Electrical energy is applied to the heart via electrodes on the leads to return the heart to normal rhythm.

A typical cardiac pacing lead is positioned within one or more chambers of the heart such that the distal end of the lead is positioned in the ventricle or atrium through a subclavian vein, while the proximal end is attached to a pacemaker which is implanted subcutaneously. Some leads also include electrodes that operate as sensors to monitor the atrium and/or ventricle of the heart, and in some applications to deliver pacing pulses to the atrium or ventricle.

One alternative cardiac pacing technique for treating tachyarrhythmias involves placing a lead in the right ventricle to pace and/or defibrillate the high septal or outflow tract of a heart from the right ventricle. Studies in the literature have demonstrated hemodynamic superiority of right ventricular outflow tract and septal pacing compared with right ventricular apical pacing.

Conventional leads are not designed for septal pacing. Accordingly, there is a need for a lead that is able to perform efficient septal pacing. The lead should be able to be implanted into the right ventricle of the heart such that electrodes on the lead can provide cardio therapy to the septum.

### SUMMARY

A lead assembly that includes a body with a proximal end and a distal end and at least one conductor disposed within the body. A distal electrode is at the distal end of the body and is electrically coupled with at least one of the conductors. A first electrode is on the body and is electrically coupled with at least one of the conductors. The lead assembly further includes a neck that is less stiff than the body. The neck is between the distal end of the body and the first electrode assembly. A fixation mechanism at the distal end of the body is capable of securing the lead assembly to a interventricular septum, or outflow tract, in a heart.

The lead assembly can be used to supply septal therapy to a patient that is suffering some heart ailment, such as a tachyarrhythmia. In some embodiments, a catheter can be used to insert the lead assembly into the right ventricle of a heart. Once the distal end of the lead assembly is attached to an upper portion of the septum, the catheter is removed.

The relatively low stiffness of the neck allows the neck to drop down along the septum or right ventricle such that any electrodes on the neck and body of the lead assembly make good conforming contact with the septum and right ventricle. The benefits of better contact between electrodes that provide therapy to the heart and the tissue of the heart are known to those of ordinary skill in the art.

In another embodiment, a lead assembly includes a body with a proximal end and a distal end and at least one conductor disposed within the body. A distal electrode is at the distal end of the body and is electrically coupled with at least one of the conductors. A first electrode is on the body and is electrically coupled with at least one of the conductors. The lead assembly further includes a neck that is less stiff than the body. The neck is between the distal end of the body and the first electrode and has a length between about 10 mm and about 30 mm.

In another embodiment, a method is provided which includes advancing a lead assembly using a catheter until the lead assembly is near tissue in a heart, such as a septum. The lead assembly includes a body and a neck that is less stiff than the body. The method further includes affixing a distal end of the body to the septum and removing the catheter to allow at least a portion of the neck to drop against the septum.

Since the neck of the lead assembly is less stiff than the body, the neck drops down against a septum in a heart once whatever insertion device that is used to attach the distal end of the lead assembly to the septum is removed from the heart. Depending on the number, type and location of any electrodes on neck and body of the lead assembly, any number of different types of cardio therapies can be provided to the septum, outflow tract and/or right ventricle (among other areas) of the heart.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is a plan view illustrating a portion of a lead assembly.

FIG. 2 is an enlarged plan view illustrating the tip of the lead assembly shown in FIG. 1.

FIG. 3 is a plan view illustrating another embodiment of a lead assembly.

FIG. 4 is a block diagram illustrating a method in accordance with another embodiment of the invention.

FIG. 5 illustrates the lead assembly of FIG. 1 inserted into a heart using a catheter.

FIG. 6 illustrates the catheter partially removed from the lead assembly of FIG. 5.

FIG. 7 illustrates the catheter removed from the lead assembly of FIG. 5.

FIG. 8 is a plan view illustrating another embodiment of a lead assembly.

FIG. 9 is an enlarged section view illustrating a portion of the lead assembly of FIG. 8.

### DETAILED DESCRIPTION

In the following detailed description, reference is made to the accompanying drawings which show by way of illustration specific embodiments in which the invention may be practiced. These embodiments are described in sufficient detail to enable those skilled in the art to practice the invention. It is to be understood that other embodiments may be utilized and that structural changes made such that the following detailed description is not to be taken in a limiting sense.

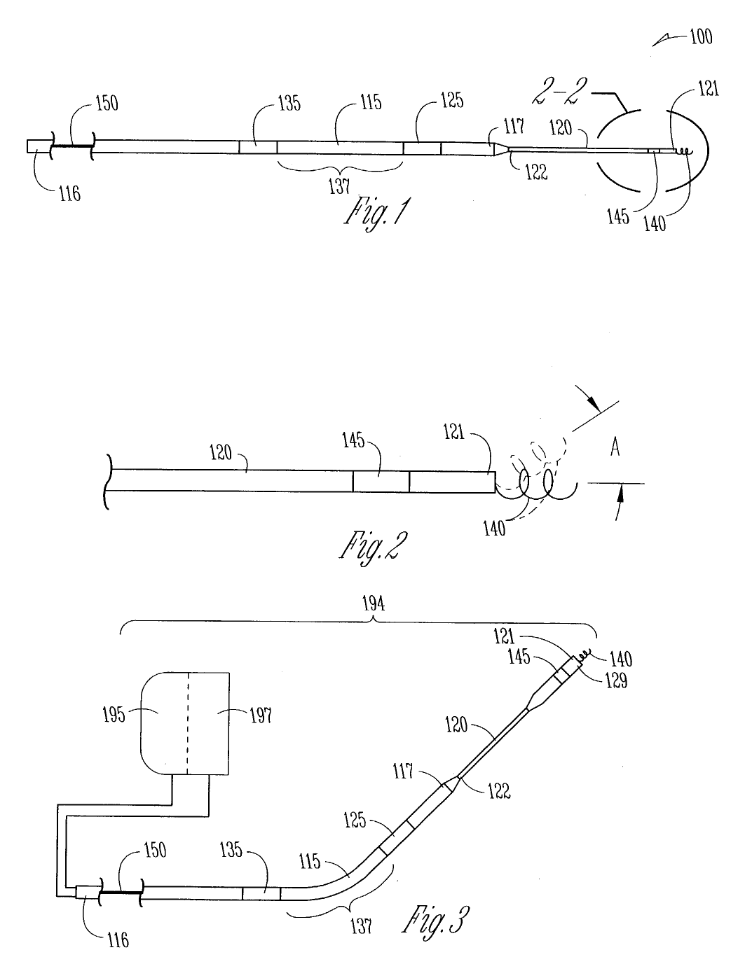

FIG. 1 illustrates a lead assembly 100 that includes a body 115 and at least one conductor 150 contained within the body 115. The lead assembly 100 is at least partially formed of a polymer, such as a medical grade silicone rubber for translumenal insertion and access within a living organism such as a patient. The conductor(s) 150 may be any form of conductor, such as, but not limited to, a cable, coil or braided conductor.

The body 115 includes a proximal end 116 and a distal end 121. The lead assembly 100 further includes a neck 120 that is less stiff than the body 115. The neck 120 is between the distal end 121 and a first electrode 125 on the body 115. In some embodiments, the neck 120 has length between about 10 mm and about 30 mm.

In the illustrated example embodiment, the first electrode 125 is electrically coupled with the at least one conductor 150 at a section 117 adjacent to an end 122 of the neck 120. The first electrode 125 is in the form of a shocking coil, although other types of electrodes may be used.

In some embodiments, the lead assembly 100 includes a second electrode 135 that is also electrically coupled with the at least one conductor 150 and is positioned between the proximal end 116 of the body 115 and the first electrode 125. The second electrode 135 is in the form of a shocking coil, although other types of electrodes may be used.

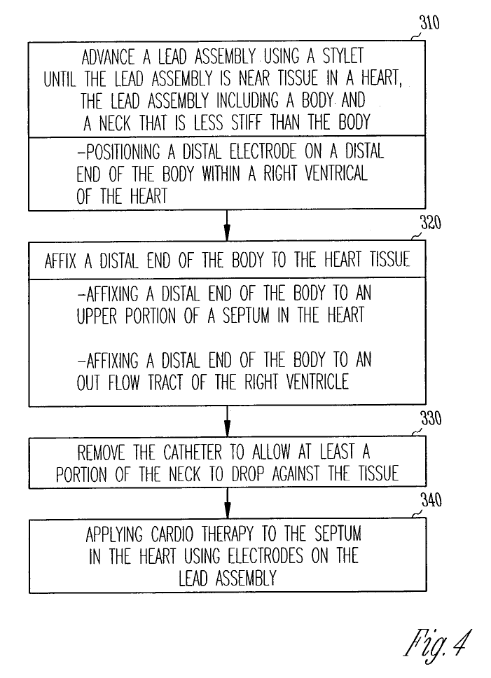

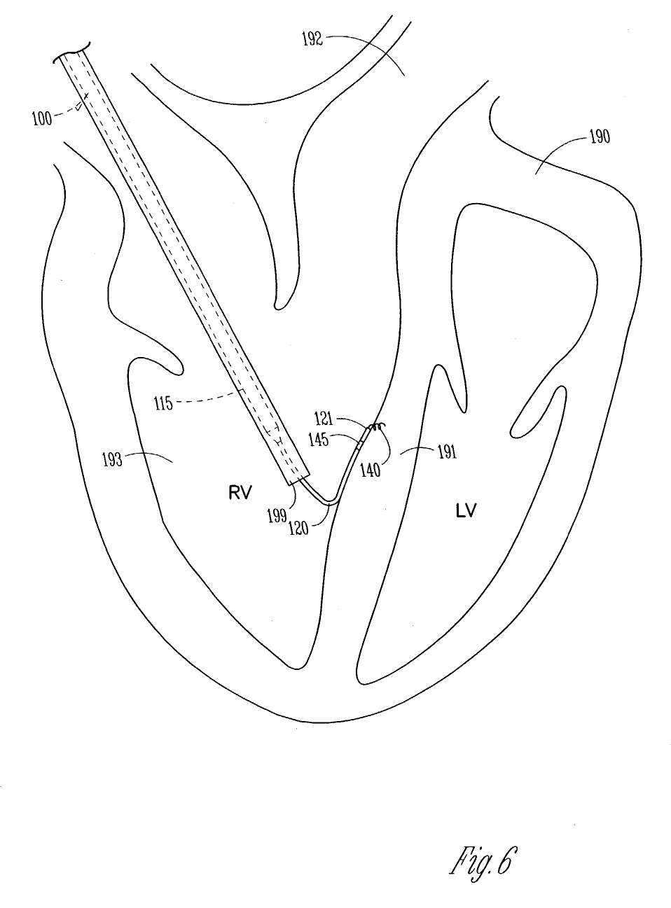

The distal end 121 of the body 115 includes a fixation mechanism, such as a helical coil 140, that is capable of securing the lead assembly 100 to tissue in a heart 190. Although the helical coil 140 is shown in FIG. 1 as being substantially aligned with the longitudinal axis of the assembly 100, the helical coil 140 may be positioned at an angle relative to the longitudinal axis of lead assembly 100 in order to facilitate attachment of the helical coil 140 to tissue of a heart 190, such as the septum 191 and/or the out flow tract 192 of a right ventricle 193 (see also FIGS. 5-7). One example is shown in phantom in FIG. 2 where the helical coil 140 is at an angle A relative to the longitudinal axis of the helical coil 140. In some embodiments, angle A is between about 0 and about 90 degrees.

The relatively low stiffness of the neck 120 on the lead assembly 100 allows the lead assembly 100 to be used to apply septal therapy to a patient that is suffering some heart ailment, such as a tachyarrhythmia. The lead assembly 100 is effective in applying such therapy because the neck 120 is adapted to drop against a septum 191 in a heart 190 once a catheter 199 that has been used to insert the lead assembly 100 into the septum 191 is withdrawn from the lead assembly 100.

All, or some, portions of the body 115 and 120 may be coated with an anti-thrombous agent, such as, but not limited to, heparin and polyethxlene glycol. The anti-thrombous agents help to alleviate fibrotic attachment between the lead assembly 100 and heart tissue.

In the illustrated example embodiment, a distal electrode 145 is electrically coupled with the at least one conductor 150 and positioned at the distal end 121 of the neck 120. Distal electrode 145 is in the form of a pacing electrode, although other types of electrodes may be used.

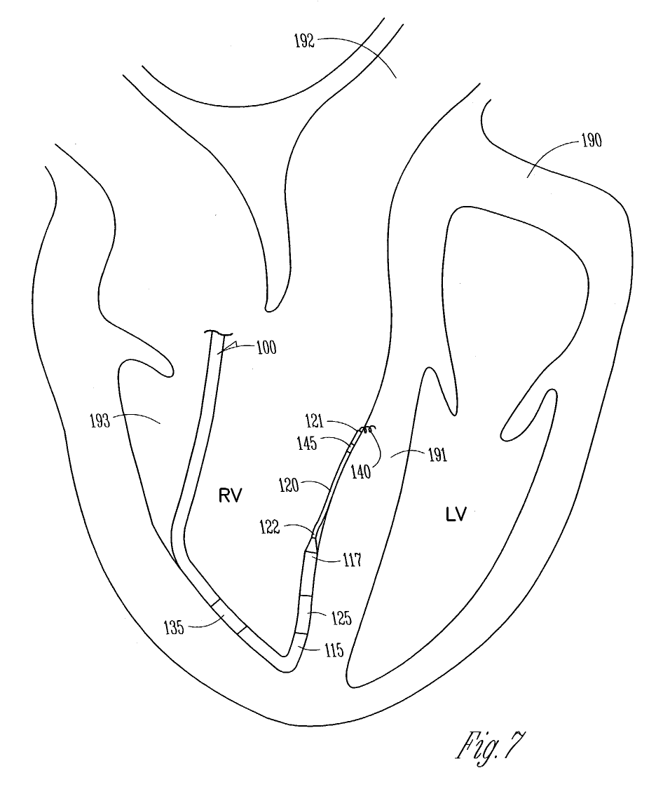

In the embodiment illustrated in FIG. 1, a section 137 of the body 115 between the first electrode 125 and the second electrode 135 is straight. In the embodiment shown in FIG. 3, section 137 is pre-formed into an arc-shape to facilitate appropriate placement of electrodes assemblies 125, 135 against tissue, such as a septum 191 and/or right ventricle 193 within a heart 190 (see, e.g., FIG. 7).

In the embodiment illustrated in FIG. 1, the neck 120 is less stiff than the body 115 because the neck 120 has a cross-sectional area that is smaller than the cross-sectional area of the body 115. In the example embodiment shown in FIG. 3, the distal end 121 of the body 115 includes a section 129 having a cross-sectional area that is larger than the cross-sectional area of the neck 120. In some embodiments, the cross-sectional area of the section 129 is substantially the same as the cross-sectional area of the body 115 (see FIG. 3).

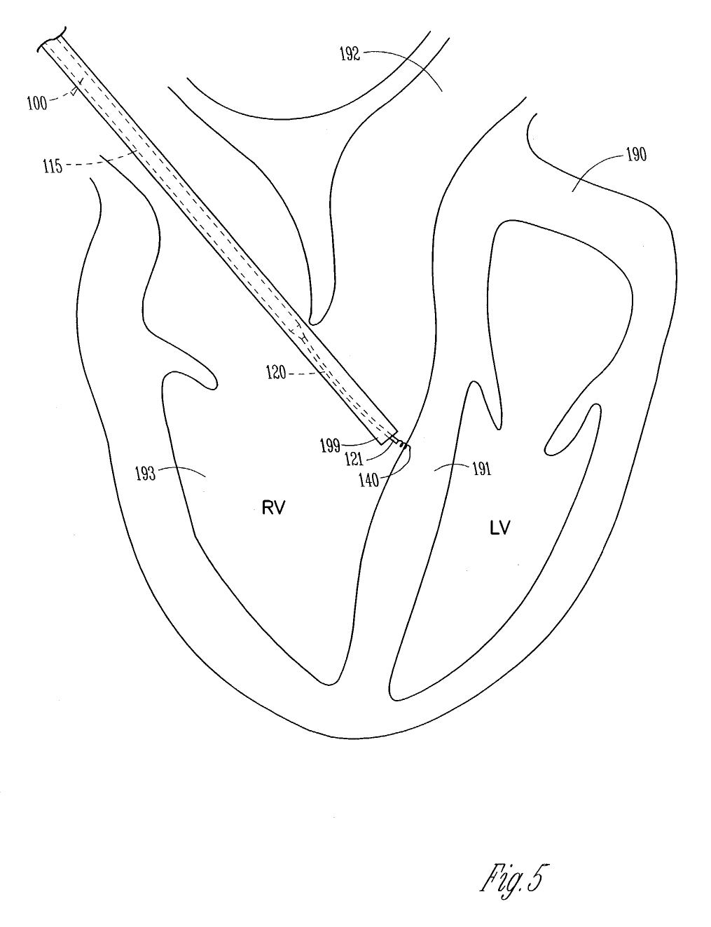

FIG. 4 shows a block diagram illustrating a method 300. The method 300 includes advancing a lead assembly using a catheter until the lead assembly is near tissue in a heart, such as a septum, the lead assembly including a body and a neck that is less stiff than the body 310; affixing a distal end of the body to the heart tissue 320; and removing the catheter to allow at least a portion of the neck to drop against the tissue 330. In some embodiments, the method further includes applying cardio therapy to the septum in the heart using electrodes on the lead assembly 340.

In some embodiments, advancing a lead assembly using a catheter 199 includes positioning the distal end of the body within a right ventricle of the heart. In addition, affixing a distal end of the body to the heart includes affixing a distal end of the body to an upper portion of a septum and/or outflow tract in the heart.

The insertion of lead assembly 100 within heart 190 will be explained further with reference to FIGS. 5-7. A catheter 199 is used to guide the lead assembly 100. The catheter 199 is manipulated to facilitate the insertion of the lead assembly 100 into and through a vein and then through an intracardiac valve. The distal end 121 of the lead body 115 is advanced into the right ventricle 193 of a heart 190 until the distal end 121 is positioned near the septum 191, or outflow tract 192 adjoining the right ventricle 193, of heart 190. In some example embodiments, the distal end of the catheter 199 is curved to faciliate insertion of the lead assembly 100 into an upper portion of the septum 191, or outflow tract 192.

The distal end 121 of the body 115 is affixed to the upper portion of the septum 191 or the out flow tract 192 (see FIG. 5) depending on factors considered by the physician at the time of implantation. As an example, the lead assembly 100 is affixed to the heart 190 by manipulating the catheter 199 to maneuver the helical coil 140 into an upper portion of the septum 191 tissue within the heart 190. Once the distal end 121 of the body 115 is affixed, the catheter 199 is removed such that the exposed portion of the neck 120 begins to drop against the septum 191 (see FIG. 6).

In some embodiments, the first electrodel25 and/or the second electrode 135 drop against the heart tissue (e.g., septum 191 and/or the bottom of right ventricle 193 as shown in FIG. 7). In other embodiments, second electrode 135 is spaced from first electrode 125 such that second electrode 135 is in a supraventricular location. The number and arrangement of electrodes on lead assembly 100 that drop against and engage the septum 191 and/or right ventricle 193 of heart 190 depend on where the distal end 121 of the body 115 engages the heart 190, and the relative locations of any electrodes on the lead assembly 100 relative to the neck 120 and the distal end 121 of the body 115.

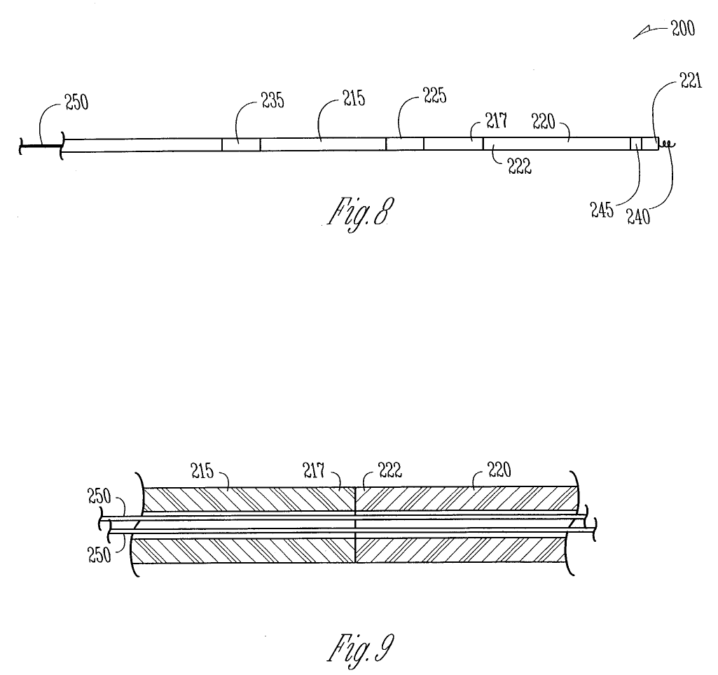

Another lead assembly 200 is shown in FIGS. 8 and 9. The lead assembly 200 similarly includes a body 215 and at least one conductor 250 (two conductors are shown in FIG. 9) contained within the body 215. The body 215 includes a proximal end (not visible in FIGS. 8 and 9) and a distal end 221. A first electrode 225 is electrically coupled with at least one of the conductors 250 and is positioned on the body 215.

The lead assembly 200 further includes a neck 220 that is less stiff than the body 215. The neck 220 is between the distal end 221 of the body 215 and the first electrode 215. The first electrode 225 is positioned on the body 215 at a section 217 that is adjacent to an end 222 of the neck 220.

In some embodiments, the lead assembly 200 includes a second electrode 235 that is also electrically coupled with at least one of the conductors 250, and is positioned between the proximal end of the body 215 and the first electrode 225.

The distal end 221 of the body 215 includes a fixation mechanism, such as a helical coil 240, that is capable of securing the lead assembly 200 to tissue in a heart 190, such as a septum 191. A distal electrode 245 is electrically coupled to at least one of the conductors 250 and is positioned at the distal end 221 of body 215.

In the example embodiment illustrated in FIGS. 8 and 9, the neck 220 is similarly less stiff than the body 215 because the neck 220 is made from a first material that is less stiff than a second material that forms the body 215. One example of the first material that forms the neck 220 is silicone, polyurethane or other biocompatible polymers. In addition, one example of the second material that forms the body 215 is silicone, polyurethane or other biocompatible polymers. In any embodiment, the neck and body of the lead assembly may be integral with one another or fabricated separately and joined together as long as the neck is less stiff than the body.

It should be noted that the lead assembly is meant to encompass all embodiments of a lead assembly that includes a neck and a body where the neck is less stiff than the body. Since the neck of the lead assembly less stiff than the body, the neck can drop against the septum when an insertion device that is used to attach the lead assembly to the septum is removed from the heart. The leads shown and described herein are able to provide any number of different types of cardio therapies to a septum, outflow tract and/or right ventricle (among other areas) of a heart.

The lead assembly 100 may also be part of a system 194 for delivering electrical pulses to stimulate a heart and/or for receiving electrical pulses to monitor the heart. The system 194 (see FIG. 3) includes a pulse generator 195 and/or signal sensor 197. As discussed above, the distal end 121 is adapted for implantation within the heart of a patient and the proximal end 116 is connected directly or indirectly to a terminal connector that electrically connects the various electrodes and conductors within the lead body to the pulse generator 195 and/or signal sensor 197. The pulse generator 195 and/or signal sensor 197 may be implanted pectorally, abdominally, or elsewhere within a patient.

It is to be understood that the above description is intended to be illustrative, and not restrictive. For instance, the leads described above include, but are not limited to, tachy or brady leads. It should be noted that features of the various above-described embodiments may be interchanged to form additional combinations. Many other embodiments will be apparent to those of skill in the art upon reviewing the above description. The scope of the invention should be determined with reference to the appended claims.

What is claimed is:

1. A lead assembly comprising:
a body including a proximal end and a distal end;
at least one conductor disposed within the body;
a distal electrode at the distal end of the body, the distal electrode electrically coupled with at least one of the conductors;
a first electrode on the body, the first electrode electrically coupled with at least one of the conductors;
a neck between the distal end of the body and the first electrode assembly, the neck being less stiff than the body; and
a fixation mechanism at the distal end of the body, the fixation mechanism capable of securing the lead assembly to a septum in a heart.

2. The lead assembly of claim 1, wherein the neck is adapted to drop down against the septum such that the distal electrode and the first electrode engage the septum.

3. The lead assembly of claim 2, further comprising a second electrode coupled with at least one of the conductors, the second electrode positioned between the proximal end of the body and the first electrode assembly.

4. The lead assembly of claim 3, wherein the neck is adapted to drop down against the septum such that the second electrode engages the right ventricle.

5. The lead assembly of claim 3, wherein a section of the body between the first electrode and the second electrode is arc-shaped.

6. The lead assembly of claim 1, wherein the fixation mechanism is a helical coil.

7. The lead assembly as recited in claim 6, wherein the helical coil is aligned with a longitudinal axis of the body.

8. The lead assembly as recited in claim 6, wherein the helical coil is at an angle between about 0 and 90 degrees relative to a longitudinal axis of the body.

9. A lead assembly comprising:
a body including a proximal end and a distal end;
at least one conductor disposed within the body;
a distal electrode at the distal end of the body, the distal electrode electrically coupled with at least one of the conductors;
a first electrode on the body, the first electrode electrically coupled with at least one of the conductors;
a neck between the distal end of the body and the first electrode assembly, the neck being less stiff than the body and including a length between about 10 mm and about 30 mm.

10. The lead assembly as recited in claim 9, wherein the body has a first cross-sectional area and the neck has a second cross-sectional area that is smaller than the first cross-sectional area of the body.

11. The lead assembly as recited in claim 10, wherein the distal end of the neck includes a section that has a third cross-sectional area which is different than the second cross-sectional area.

12. The lead assembly as recited in claim 11, wherein the third cross-sectional area of the section at the distal end of the neck is substantially the same size as the first cross-sectional area of the body.

13. The lead assembly as recited in claim 9, wherein the body is made of a first material and the neck is made of a second material.

14. The lead assembly as recited in claim 9, wherein the body coated with an anti-thrombous agent.

15. The lead assembly as recited in claim 9, wherein the first electrode is a shocking coil.

16. The lead assembly as recited in claim 9, wherein the distal electrode is a sensing electrode.

17. A method comprising:
advancing a lead assembly using a catheter until the lead assembly is near tissue in a heart, the lead assembly including a body and a neck that is less stiff than the body;
affixing a distal end of the body to the heart tissue; and
removing the catheter to allow at least a portion of the neck to drop against the tissue.

18. The method as recited in claim 17, wherein advancing a lead assembly using a catheter includes positioning the distal end of the body within a right ventricle of the heart, and affixing a distal end of the body to the heart tissue includes affixing a distal end of the body to an upper portion of a septum in the heart.

19. The method as recited in claim 17, wherein affixing a distal end of the body to an upper portion of a septum in the heart includes affixing a distal end of the body to an outflow tract of the right ventricle.

20. The method as recited in claim 19, further comprising applying cardio therapy to the septum in the heart using electrodes on the lead assembly.

21. A lead assembly comprising:
a body including a proximal end and a distal end;
at least one conductor disposed within the body;
a distal electrode at the distal end of the body, the distal electrode electrically coupled with at least one of the conductors;
a first electrode on the body, the first electrode electrically coupled with at least one of the conductors;
a second electrode located on the body between the proximal end of the body and the first electrode assembly, the second electrode electrically coupled with at least one of the conductors;
a neck between the distal end of the body and the first electrode assembly, the neck being less stiff than the body and including a length between about 10 mm and about 30 mm; and
a fixation mechanism at the distal end of the body, the fixation mechanism capable of securing the lead assembly to a septum in a heart.

22. The lead assembly as recited in claim 21, further comprising a pulse generator electrically coupled to at least one of the conductors.

23. The lead assembly as recited in claim 21, further comprising a signal sensor electrically coupled to at least one of the conductors.
